(19) United States
(12) Patent Application Publication
(10) Pub. No.: US 2007/0226813 A1
(43) Pub. Date: Sep. 27, 2007

(54) METHODS OF SCREENING FOR ANTITUMOR AGENTS

(75) Inventors: Hendrik Luesch, Gainesville, FL (US); Sumit Chanda, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); THE SCRIPPS RESEARCH INSTITUTE, La Jolla, CA (US)

(21) Appl. No.: 11/668,873

(22) Filed: Jan. 30, 2007

Related U.S. Application Data
(63) Continuation of application No. PCT/US05/26927, filed on Jul. 28, 2005.
(60) Provisional application No. 60/592,841, filed on Jul. 30, 2004.

(51) Int. Cl. G01N 33/53; G01N 33/00

(52) U.S. Cl. 800/3; 435/7.23

(57) ABSTRACT

This invention provides cellular regulators of antitumor agent apratoxin A. The invention also provides methods for identifying novel antitumor compounds using these cellular regulators of apratoxin A. The methods comprise first screening test agents for modulators of a cellular regulator of apratoxin A and then further screening the identified modulating agents for antitumor activities. The invention further provides methods and pharmaceutical compositions for treating tumors in a subject.

### CROSS-REFERENCE TO RELATED APPLICATIONS

This application claims the benefit of priority to PCT International Application No. PCT/U.S. 2005/026927, filed Jul. 28, 2005 and U.S. Provisional Patent Application No. 60/592,841, filed Jul. 30, 2004. The full disclosure of these applications is incorporated herein by reference in its entirety and for all purposes.

### FIELD OF THE INVENTION

The present invention generally relates to identification of novel drug targets and to methods of screening for antitumor agents using such novel targets. More particularly, the invention pertains to cellular regulators of antitumor agent apratoxins (esp. apratoxin A) and to methods of using these cellular regulators to identify novel antitumor compounds.

### BACKGROUND OF THE INVENTION

Apratoxins are natural marine products which exhibit potent cytotoxicity against a variety of human tumor cell lines. See, e.g., Luesch et al., J. Am. Chem. Soc. 123: 5418-5423, 2001; and Luesch et al., Bioorg. Med. Chem. 10: 1973-1978, 2002. Apratoxin A is the most potent compound of this family identified thus far. It has a unique differential cytotoxicity profile in the NCI's 60-cell line panel. The mechanism of action of apratoxin A, including its cellular targets, remains unknown.

Identification of the cellular regulators of apratoxins, especially apratoxin A, will lead to a better understanding of mechanism of apratoxin mediated tumor cytotoxicity. These molecules would also provide targets to screen for and develop novel antitumor agents. The instant invention fulfills this and other needs in the art.

### SUMMARY OF THE INVENTION

The present invention relates to identification of cellular regulators of antitumor agent apratoxin A, methods for screening novel antitumor compounds using these cellular regulators, and methods for treating tumors using such antitumor compounds.

In one aspect, the invention provides methods for identifying novel antitumor agents. The methods entail (a) assaying a biological activity of a cellular regulator of apratoxin A-induced apoptosis in the presence of a test agent to identify one or more modulating agents that modulate the biological activity of the cellular regulator; and (b) testing one or more of the modulating agents for antitumor cytotoxic activity. In some of the methods, the cellular regulator is encoded by a polynucleotide selected from the members listed in Table 2. In some methods, the assaying of the biological activity of the polypeptide occurs in vitro. In some methods, the biological activity is protein kinase activity and the cellular regulator is Prkaca, RIOK2, or CLK3. In some other methods, the biological activity is protein phosphatase activity and the polypeptide is Ppp 1cc.

In some of the methods, the biological activity assayed is a specific binding of the test agent to the cellular regulator of apratoxin A-induced apoptosis. In some methods, the test agent inhibits the biological activity of the cellular regulator. In some other methods, the test agent stimulates the biological activity of the cellular regulator. In some methods, the test agent modulates cellular level of the cellular regulator.

In some methods of the invention, the modulating agents are screened for ability to inhibit proliferation of a tumor cell in vitro. For example, a cultured tumor cell line can be employed in the screening. In some methods, a human solid tumor cell line such as KB or LoVo is used. Some of the methods further include a control test to examine the modulating agents for cytotoxicity on a non-tumor control cell. In some methods, the modulating agents are tested for antitumor activity on a tumor in an animal.

In a related aspect, the invention provides methods for identifying compounds that inhibit tumor cell proliferation. The methods involve (a) contacting a test agent with a cellular regulator of apratoxin A-induced apoptosis to identify one or more modulating agents that modulate a biological activity of the cellular regulator; and (b) detecting a reduced proliferation of a tumor cell in the presence of the modulating agent relative to proliferation of the tumor cell in the absence of the test agent. The modulating agents can be examined for antitumor activities in vitro using tumor cell line (e.g., human solid tumor cell line KB or LoVo). A control test with a non-tumor cell line can also be included in the methods.

### DETAILED DESCRIPTION

I. Overview

The invention is predicated in part on the discovery by the present inventors of cellular regulators of small molecule antitumor agents such as apratoxin A. The discovery was based on genome-wide overexpression screens in mammalian cells for targets identification and biological mechanism studies.

Specifically, a genome-wide phenotypic complementation strategy was employed by the present inventors to identify cDNAs that are able to rescue cells from apratoxin A-induced apoptosis. This was accomplished by using an arrayed collection of full-length expression cDNAs (˜27,000 clones). Specifically, individual genes in the cDNA matrix were transfected into U2OS cancer cells utilizing high-throughput methodology. A constitutively active luciferase reporter was cotransfected as indicator of cell viability. Screens were run in the absence and presence (˜IC90) of apratoxin A. Statistical analysis revealed cDNAs which confer resistance to apratoxin A. Those cDNA hits were assessed for their effect on the dose-response curve and cell cycle profile.

In accordance with these discoveries, the present invention provides methods for identifying novel antitumor agents. The invention also provides methods for inhibiting tumorigenesis and proliferation of tumor cells, and methods for treating various tumors. The following sections provide guidance for making and using the compositions of the invention, and for carrying out the methods of the invention.

II. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., Dictionary of Microbiology and Molecular Biology (3d ed. 2002); the Larousse Dictionary of Science and Technology (Walker ed., 1995); and the Collins Dictionary of Biology (2d ed. 1999). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule (e.g., a cellular regulator of apratoxin A-induced apoptosis or a binding ligand) but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., a test compound and a protein target) or combining agents and cells (e.g., a protein and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “a cellular regulator of apratoxin A-induced apoptosis” or “a cellular regulator of apratoxin A” used herein refers broadly to proteins and polypeptides that directly or indirectly interact with apratoxin A to modulate its cytotoxic activity. It includes two broad classes of molecules: (i) cellular proteins (e.g., a binding target of apratoxin A) that positively participate in (facilitate or stimulate) apratoxin A-mediated cytotoxicity and (ii) molecules that negatively impact (e.g., suppress or inhibit) cytotoxic activity of apratoxin A. The modulatory effects of both classes of molecules can be either due to a direct interaction with apratoxin A, or due to an indirect interaction by interacting with (e.g., binding to and/or modulating) another molecule which otherwise modulates apratoxin A-mediated cytotoxicity. As demonstrated in the Example below, overexpression of a member of both classes of these cellular regulators in a cell (e.g., a tumor cell) lead to an inhibition of apratoxin A-induced apoptosis. However, different mechanisms might underlie the inhibition. For the first class, recombinant overexpression of a regulator of the first class in the host cell could lead to inhibition of apratoxin A-induced apoptosis by saturating apratoxin A. On the other hand, overexpression of a member in the second class likely results in the inhibition through compensatory mechanisms.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein and are generally available.

A “host cell,” as used herein, refers to a prokaryotic or eukaryotic cell that contains heterologous DNA that has been introduced into the cell by any means, e.g., electroporation, calcium phosphate precipitation, microinjection, transformation, viral infection, and/or the like.

The terms “identical” or “identical sequences” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In some embodiments, the polypeptides to be employed in the present invention can have at least 70%, generally at least 75%, optionally at least 80%, 85%, 90%, 95% or 99% or more identical to a reference polypeptide, e.g., a cellular regulator of apratoxin A-induced apoptosis encoded by a polynucleotide in Table 1 or 2. The percentage of sequence identity can be measured, e.g., by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., as measured by BLASTN (or CLUSTAL, or any other available alignment software) using default parameters.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “modulate” with respect to a cellular regulator of apratoxin A-induced apoptosis refers to a change in its cellular level or other biological activities (e.g., kinase activity). For example, modulation may cause an increase or a decrease in cellular levels of the cellular regulator, enzymatic modifications (e.g., phosphorylation), or any other biological, functional, or immunological properties of such proteins. The change in activity can arise from, for example, an increase or decrease in expression of one or more genes that encode the cellular regulator, the stability of an mRNA that encodes the cellular regulator, translation efficiency, or from a change in a biological activity of the cellular regulator itself. The change can also be due to the activity of another molecule that modulates a cellular regulator of apratoxin A.

Modulation of activities of a cellular regulator of apratoxin A-induced apoptosis can be up-regulation (i.e., activation or stimulation) or down-regulation (i.e. inhibition or suppression). The mode of action can be direct, e.g., through binding to the cellular regulator or to genes encoding the cellular regulator, or indirect, e.g., through binding to and/or modifying (e.g., enzymatically) another molecule which otherwise modulates the cellular regulator (e.g., an enzyme which acts on the cellular regulator of apratoxin A).

The term “polypeptide” is used interchangeably herein with the term “protein”, and refers to a polymer of amino acid residues, e.g., as typically found in proteins in nature. A “mature protein” is a protein which is full-length and which, optionally, includes glycosylation or other modifications typical for the protein in a given cell membrane.

A “variant” of a molecule such as a cellular regulator of apratoxin A-induced apoptosis or a binding ligand is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

III. Screening for Novel Antitumor Agents

A. Cellular Regulators of Apratoxin A-Induced Apoptosis and Screening Scheme

The present invention provides cellular regulators of apratoxin A-induced apoptosis. These molecules when overexpressed in host cells confer resistance to apratoxin A induced apoptosis. As detailed in Example 1 below, a number of polynucleotides were identified which were able to rescue a human cancer cell line from apratoxin A-induced apoptosis when the polynucleotides were transfected into the host cell. Exemplary polynucleotides encoding such cellular regulators of apratoxin A-induced apoptosis are shown in Tables 1 and 2. As shown in the Tables, the novel cellular regulators of apratoxin A-induced apoptosis include very diverse classes of proteins, including kinases, phosphatases, RNA-binding proteins, and receptor-binding polypeptides.

The cellular regulators of apratoxin A-induced apoptosis identified by the present inventors provide novel targets to screen for antitumor agents. Employing the these cellular regulators of apratoxin A described herein, the present invention provides methods for screening novel antitumor agents or compounds that function by modulating activities of a cellular regulator of apratoxin A. Various biochemical and molecular biology techniques or assays well known in the art can be employed to practice the present invention. Such techniques are described in, e.g., Sterling et al., Methods and Techniques in Drug Discovery, Mary Ann Liebert, New York (2004); Atterwill et al., Approaches to High Throughput Toxicity Screening, CRC Press (1999); William P. Janzen, High Throughput Screening: Methods and Protocols (Methods in Molecular Biology, 190), Humana Press (2002); Sambrook et al., Molecular Cloning: A Laboratory Manual, Cold Spring Harbor Press, N.Y., 3^{rd} ed. (2000) Editions; and Ausubel et al., Current Protocols in Molecular Biology, John Wiley & Sons, Inc., New York (1987-1999).

Typically, test agents are first assayed for their ability to modulate a biological activity of a cellular regulator of apratoxin A-induced apoptosis (“the first assay step”). Modulating agents thus identified are then subject to further screening for antitumor activities, typically in the presence of the cellular regulator (“the second testing step”). Depending on the cellular regulator of apratoxin A employed in the method, modulation of different biological activities of the cellular regulator can be assayed in the first step. For example, a test agent can be assayed for binding to the cellular regulator. The test agent can be assayed for activity to modulate expression level of the cellular regulator, e.g., transcription or translation. The test agent may also be assayed for activities in modulating cellular level or stability of the cellular regulator, e.g., post-translational modification or proteolysis.

If the cellular regulator of apratoxin A-induced apoptosis has a known biological or enzymatic function (e.g., kinase activity, phosphatase activity, or RNA-binding activity), the biological activity monitored in the first screening step can be the specific biochemical or enzymatic activity. For example, enzymatic activity of a kinase (e.g., Prkaca, RIOK2 or CLK3 in Table 2) or a phosphatase (e.g., Ppp 1cc in Table 2) can be monitored in the first screening step if any of these cellular regulators of apratoxin A-induced apoptosis is employed in the screening. In an exemplary embodiment, the cellular regulator is a kinase (e.g., Prkaca, RIOK2 or CLK3 in Table 2), and test agents are first screened for modulating the kinase's activity in phosphorylating a substrate. Methods for assaying such biological activities (e.g., kinase activity or phosphatase activity) are well known and routinely practiced in the art. The substrate can be a molecule known to be enzymatically modified by the cellular regulator, or a molecule that can be easily identified from candidate substrates for a given class of enzymes. For example, many kinase substrates are available in the art. See, e.g., www.emdbiosciences.com; and www.proteinkinase.de.

In addition, a suitable substrate of a kinase can be screened for in high throughput format. For example, substrates of a kinase can be identified using the Kinase-Glo® luminescent kinase assay (Promega) or other kinase substrate screening kits (e.g., developed by Cell Signaling Technology, Beverly, Mass.). Similarly, substrates of a phosphatase can be identified using assays well known in the art. For example, many protein kinase and phosphatase-related assays are described in Methods in Enzymology, Vol. 200, Tony Hunter (ed.), Academic Press, New York, 1991; Protein Phosphatase Protocols, John W. Ludlow, Humana Press, 1998; and Methods in Enzymology, Vol. 366, Susanne Klumpp (ed.), Academic Press, New York, 2003.

As noted above, the cellular regulators of apratoxin A-induced apoptosis include both positive and negative regulators. Therefore, test agents can be screened for ability to either up-regulate or down-regulate a biological activity in the first assay step. Once test agents that modulate a biological activity of the cellular regulator of apratoxin A are identified, they are typically further tested for cytotoxic activity against tumor cells. This further testing step is often needed to confirm that their modulatory effect on the cellular regulator would indeed lead to cytotoxicity of tumor cells. For example, a test agent which modulates phosphorylation activity of a cellular regulator of apratoxin A needs to be further tested in order to confirm that such modulation can result in cytotoxic effects in tumor cells. In some embodiments, the modulating agents identified from the first screening step are further examined for any cytotoxicity in non-tumor cells. This additional step could ensure that the antitumor agents identified with the screening methods of the invention are specific for tumor cells.

In both the first assaying step and the second testing step, either an intact cellular regulator of apratoxin A or a fragment thereof may be employed. Analogs or functional derivatives of the cellular regulator could also be used in the screening. The fragments or analogs that can be employed in these assays usually retain one or more of the biological activities of the cellular regulator (e.g., kinase activity if the cellular regulator employed in the first assaying step is a kinase). Fusion proteins containing such fragments or analogs can also be used for the screening of test agents. Functional derivatives of a cellular regulator of apratoxin A usually have amino acid deletions and/or insertions and/or substitutions while maintaining one or more of the bioactivities and therefore can also be used in practicing the screening methods of the present invention. A functional derivative can be prepared from a cellular regulator of apratoxin A by proteolytic cleavage followed by conventional purification procedures known to those skilled in the art. Alternatively, the functional derivative can be produced by recombinant DNA technology by expressing only fragments of a cellular regulator of apratoxin A that retain one or more of their bioactivities.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occuring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of a cellular regulator of apratoxin A. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the cellular regulators of apratoxin A-induced apoptosis discussed above. Such structural studies allow the identification of test agents that are more likely to bind to the cellular regulators of apratoxin A-induced apoptosis. The three-dimensional structures of the cellular regulators of apratoxin A-induced apoptosis or an apratoxin A subunit can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of structures of the cellular regulators of apratoxin A-induced apoptosis provides another means for designing test agents for screening. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system.” In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, N.Y. 1986).

Modulators of the present invention also include antibodies that specifically bind to a cellular regulator of apratoxin A-induced apoptosis in Table 1 or 2. Such antibodies can be monoclonal or polyclonal. Such antibodies can be generated using methods well known in the art. For example, the production of non-human monoclonal antibodies, e.g., murine or rat, can be accomplished by, for example, immunizing the animal with a cellular regulator of apratoxin A or its fragment (see, e.g., Harlow & Lane, Antibodies, A Laboratory Manual, Cold Spring Harbor Laboratory Press, Cold Spring Harbor, N.Y., 3^{rd} ed., 2000). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a cellular regulator of apratoxin A.

Human antibodies against a cellular regulator of apratoxin A can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a cellular regulator of apratoxin A or its fragment.

C. Screening for Compounds that Modulate a Cellular Regulator of Apratoxin A

Typically, the test agents are first screened for ability to modulate a biological activity of a cellular regulator of apratoxin A identified by the present inventors. Unless otherwise specified, modulation of a biological activity of a cellular regulator of apratoxin A includes modulation of its cellular as well as other biological or cellular activities. A number of assay systems can be employed in the first screening step to screen test agents for modulators of a cellular regulator of apratoxin A. The screening can utilize an in vitro assay system or a cell-based assay system. In this screening step, test agents can be screened for binding to the cellular regulator, altering cellular level of the cellular regulator, or modulating other biological activities of the cellular regulator of apratoxin A.

1. Binding of Test Agents to a Cellular Regulator of Apratoxin A

In some methods, binding of a test agent to a cellular regulator of apratoxin A-induced apoptosis is determined in the first screening step. Binding of test agents to a cellular regulator of apratoxin A can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the cellular regulator, e.g., co-immunoprecipitation with the cellular regulator of apratoxin A by an antibody directed to the cellular regulator. The test agent can also be identified by detecting a signal that indicates that the agent binds to the cellular regulator, e.g., fluorescence quenching.

Competition assays provide a suitable format for identifying test agents that specifically bind to a cellular regulator of apratoxin A-induced apoptosis. In such formats, test agents are screened in competition with a compound already known to bind to the cellular regulator of apratoxin A. The known binding compound can be a synthetic compound. It can also be an antibody, which specifically recognizes the cellular regulator, e.g., a monoclonal antibody directed against the cellular regulator. If the test agent inhibits binding of the compound known to bind the cellular regulator, then the test agent also binds the cellular regulator of apratoxin A.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see, Harlow and Lane, “Antibodies, A Laboratory Manual,” Cold Spring Harbor Press, 3^{rd} ed., 2000); solid phase direct label RIA using ¹²⁵I label (see Morel et al., Mol. Immunol. 25(1):7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize a cellular regulator of apratoxin A or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the cellular regulator, the test agents are bound to the solid matrix and the cellular regulator molecule is then added.

Soluble assays include some of the combinatory libraries screening methods described above. Under the soluble assay formats, neither the test agents nor the cellular regulator of apratoxin A are bound to a solid support. Binding of a cellular regulator of apratoxin A or fragment thereof to a test agent can be determined by, e.g., changes in fluorescence of either the cellular regulator or the test agents, or both. Fluorescence may be intrinsic or conferred by labeling either component with a fluorophor.

In some binding assays, either the cellular regulator of apratoxin A, the test agent, or a third molecule (e.g., an antibody against the cellular regulator) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Agents Modulating Other Activities of Cellular Regulators of Apratoxin A-Induced Apoptosis

Binding of a test agent to a cellular regulator of apratoxin A-induced apoptosis provides an indication that the agent can be a modulator of the cellular regulator. It also suggests that the agent may modulate biological activities of the target. Thus, a test agent that binds to a cellular regulator of apratoxin A can be further tested for ability to inhibit proliferation of a tumor cell or other antitumor activities (i.e., in the second testing step outlined above).

Alternatively, a test agent that binds to a cellular regulator of apratoxin A can be further examined to determine any effect on other biological or enzymatic functions of the cellular regulator. The existence, nature, and extent of such activity can be tested by an activity assay. Such an activity assay can confirm that the test agent binding to the cellular regulator of apratoxin A indeed has a modulatory activity on the cellular regulator. More often, such activity assays can be used independently to identify test agents that modulate activities of a cellular regulator of apratoxin A-induced apoptosis (i.e., without first assaying their ability to bind to the cellular regulator of apratoxin A).

As noted above, the cellular regulators of apratoxin A-induced apoptosis of the present invention include a very diverse class of proteins. The term “bioactivity” or “biological activity” of a cellular regulator of apratoxin A-induced apoptosis refers to any of the biochemical and physiological roles played by a cellular regulator of apratoxin A-induced apoptosis. Any of the biological activities (e.g., enzymatic activities) of a cellular regulator of apratoxin A can be tested in the presence of test compounds or compounds that have been identified to bind to the cellular regulator. Biological activities of a cellular regulator of apratoxin A-induced apoptosis to be monitored in this screening step can also include activities relating to its cellular level and enzymatic or non-enzymatic modifications.

Typically, this screening step involves adding test compounds to a sample containing a cellular regulator of apratoxin A in the presence or absence of other molecules or reagents which are necessary to test a biological activity of the cellular regulator (e.g., kinase activity if the cellular regulator is a kinase), and determining an alteration in the biological activity of the cellular regulator of apratoxin A. In an exemplary embodiment, the cellular regulator is a kinase, and the test agent is examined for ability to modulate the kinase activity of the cellular regulator. Exemplary methods for monitoring various kinase activities are described, e.g., in Chedid et al., J. Immunol. 147: 867-73, 1991; Kontny et al., Eur J Pharmacol. 227: 333-8, 1992; Wang et al., Oncogene 13: 2639-47, 1996; Murakami et al., Oncogene 14: 2435-44, 1997; Pyrzynska et al., J. Neurochem.74: 42-51, 2000; Berry et al., Biochem Pharmacol. 62: 581-91, 2001; Cai et al., Chin Med J (Engl). 114: 248-52, 2001. These methods can be employed and modified to assay modulatory effect of a test agent on a cellular regulator of apratoxin A that is a kinase (e.g., Prkaca, RIOK2 or CLK3 in Table 2).

D. Testing Modulating Agents for Antitumor Activities

Once a modulating agent has been identified to bind to a cellular regulator of apratoxin A and/or to modulate a biological activity (including cellular level) of the cellular regulator, it can be further tested for antitumor activity. Typically, this screening step is performed in the presence of the cellular regulator of apratoxin A on which the modulating agent acts. Preferably, this screening step is performed in vivo using cells that endogenously express the cellular regulator of apratoxin A. As a control, cytotoxicity of the modulating agents on cells that do not express the cellular regulator can also be examined.

A variety of human tumor cell lines can be employed in this screening step. For example, human solid tumor cell lines KB or LoVo are suitable for monitoring antitumor cytotoxicity of the modulating agents identified in the first screening step. Other tumor cells that can be used in the screening methods of the invention include the U2OS cell line as described in the Example below, or human glioblastoma cell line U373 (ATCC). These tumor cell lines, as well as methods for assaying cytotoxic activity of potential antitumor agents on these cells, are described in the art, e.g., Luesch et al., J. Am. Chem. Soc. 123: 5418-5423, 2001; and Luesch et al., Bioorg. Med. Chem. 10: 1973-1978, 2002.

In some embodiments, the tumor cells are first administered with the modulating agents identified in the first screening step. Antitumor cytotoxicity of the compounds is then examined in vitro. For example, the in vitro cytotoxicity can be monitored by measuring the IC₅₀ value (i.e., the concentration of a compound which causes 50% cell growth inhibition) of each of the modulating compounds. Preferably, an antitumor agent identified from this screening step will have an IC₅₀ value less than 1 μM on one or more of the tumor cell lines. More preferably, the IC₅₀ value of antitumor agents identified in accordance with the present invention is less than 250 nM. Some of the antitumor agents have an IC₅₀ value of less than 50 nM, less than 10 nM on at least one of the above described tumor cell lines. Most preferably, the antitumor agents obtained from this screening step will have an IC₅₀ value that is less than 1 nM. Methods of determining IC₅₀ values of compounds in inhibiting cultured cell lines are well known in the art. These were described in the art, e.g., Remington, The Science and Practice of Pharmacy, Mack Publishing Co., 20^{th} ed., 2000; Luesch et al., J. Am. Chem. Soc. 123: 5418-5423, 2001; Luesch et al., Bioorg. Med. Chem. 10: 1973-1978, 2002; and U.S. Pat. No. 6,552,027.

In some methods, tumor cells can be plated onto 96-well plates prior to admistering the compounds. Following incubation, absorbance of each well is measured with a microplate reader (e.g., using Labsystems reader at 540 nm and a reference wavelength of 690 nm). The absorbance values (e.g., OD₅₄₀ readings) are translated into the number of live cells in each well by comparing to those on standard cell number curves generated for the cell line. The IC₅₀ values can then be calculated by non-linear regression analysis.

Other than or in addition to monitoring in vitro cytotoxicity on cultured tumor cell lines, modulating agents identified in the first screen step may also be assessed for their antitumor activity in vivo. They can be administered to animals (e.g., mice) that bear naturally occurring- or implanted tumors to examine their antitumor activities. For example, mice bearing subcutaneous implanted early stage colon adenocarcinoma have been used to study in vivo cytotoxicity of apratoxin A related compounds (Luesch et al., J. Am. Chem. Soc. 123: 5418-5423, 2001). Such in vivo systems can also be employed to screen for antitumor agents in the present invention.

In some methods, the modulating agents identified in the first screening step are further tested for cytotoxicity on non-tumor control cells. This additional step is performed to identify compounds that selectively inhibit proliferation of tumor cells while having little or no effect on growth of normal cells. There are many non-tumor cell lines available in the art. Examples include human umbilical vein endothelial cell line (HUVEC); epithelial cell line MCF-10A (Soule et al., Cancer Res. 50: 6075-6086, 1990); colon cell line (CCD-18Co) and ovarian cell line (NOV-31 (Hirasawa et al., Cancer Research 62, 1696-1701, Mar. 15, 2002). These cells can be employed to screen modulating agents for selective cytotoxicity on tumor cells.

In addition, ATCC provides many tumor/normal cell line pairs that are used to elucidate the underlying causes of cancers. They can also be employed to screen modulating agents of the present invention to identify selective anti-tumor agents. These tumor/normal cell line pairs include non-small cell lung cancer cell line (ATCC No. CCL-256) and normal peripheral blood cell line ATCC No. CCL-256.1; adenocarcinoma cell line ATCC No. CRL-5868 and normal peripheral blood cell line ATCC No. CRL-5957; malignant melanoma cell line ATCC No. CRL-1974 and normal cell line ATCC No. CRL-1980; basal cell carcinoma cell line ATCC No. CRL-7762 and normal skin cell line ATCC No. CRL-7761; colorectal adenocarcinoma cell line ATCC No. CCL-228 and normal lymph node cell line ATCC No. CCL-227; and giant cell sarcoma cell line ATCC No. CRL-7554 and normal bond cell line ATCC No. CRL-7553. Any of these cell line pairs can be used to screen the modulating agents for compounds that selectively inhibit proliferation of tumor cells.

IV. Therapeutic Applications

Tumors are abnormal growths resulting from the hyperproliferation of cells. Cells that proliferate to excess but stay put form benign tumors, which can typically be removed by local surgery. In contrast, malignant tumors or cancers comprise cells that are capable of undergoing metastasis, i.e., a process by which hyperproliferative cells spread to, and secure themselves within, other parts of the body via the circulatory or lymphatic system (see, generally, Molecular Biology of the Cell, Alberts et al. (eds.), 4^{th} edition, Garland Science Publishing, Inc., New York, 2002). Employing the novel antitumor agents described, the invention provides therapeutic compositions and methods for preventing or treating various forms of tumors, benign or malignant, by targeting one or more of the cellular regulators of apratoxin A-induced apoptosis. The pharmaceutical compositions can comprise an antitumor agent identified in accordance with the present invention.

A great number of diseases and conditions are amenable to treatment with methods and compositions of the present invention. Examples of tumors that can be treated with methods and compositions of the present invention include but are not limited to skin, breast, brain, cervical carcinomas, testicular carcinomas. They encompass both solid tumors or metastatic tumors. Cancers that can be treated by the compositions and methods of the invention include cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

The antitumor targets of the present invention can be directly administered under sterile conditions to the subject to be treated. The modulators can be administered alone or as the active ingredient of a pharmaceutical composition. Therapeutic composition of the present invention can be combined with or used in association with other therapeutic agents. For example, a subject may be treated concurrently with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahay, N.J., 1987).

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, modulatory compounds or transduced cell), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the antitumor compound can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics , 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

The therapeutic formulations can be delivered by any effective means that can be used for treatment. Depending on the specific antitumor agent to be administered, the suitable means include oral, rectal, vaginal, nasal, pulmonary administration, or parenteral (including subcutaneous, intramuscular, intravenous and intradermal) infusion into the bloodstream. For parenteral administration, antitumor agents of the present invention may be formulated in a variety of ways. Aqueous solutions of the modulators may be encapsulated in polymeric beads, liposomes, nanoparticles or other injectable depot formulations known to those of skill in the art. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The therapeutic formulations can conveniently be presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of an antitumor agent of the present invention usually lies within the range of from about 0.001 to about 1000 mg, more usually from about 0.01 to about 500 mg per day. The preferred dosage and mode of administration of an antitumor agent can vary for different subjects, depending upon factors that can be individually reviewed by the treating physician, such as the condition or conditions to be treated, the choice of composition to be administered, including the particular antitumor agent, the age, weight, and response of the individual subject, the severity of the subject's symptoms, and the chosen route of administration. As a general rule, the quantity of an antitumor agent administered is the smallest dosage which effectively and reliably prevents or minimizes the conditions of the subjects. Therefore, the above dosage ranges are intended to provide general guidance and support for the teachings herein, but are not intended to limit the scope of the invention.

V. Example: Identification of Cellular Regulators of Apratoxin A-Induced Apoptosis

The following example is offered to illustrate, but not to limit the present invention. This Example describes identification of various cDNAs which upon overexpression, confer to host cells resistance to apratoxin A.

The study employs a genome-wide phenotypic complementation strategy to identify cDNAs able to rescue from apratoxin A-induced apoptosis by using an arrayed collection of full-length expression cDNAs (˜27,000 clones). Specifically, individual genes in the cDNA matrix were transfected into U2OS cancer cells (a human cancer cell line) utilizing high-throughput methodology. A constitutively active luciferase reporter was cotransfected as indicator of cell viability. Screens were run in the absence and presence (˜IC₉₀) of apratoxin A. Statistical analysis was then performed to reveal cDNAs which conferred resistance to apratoxin A. Those cDNA hits were assessed for their effect on the dose-response curve and cell cycle profile. Table 1 lists cDNAs that confer resistance to apratoxin A upon the U2OS cancer cells. Proteins encoded by these cDNAs represent cellular regulators of apratoxin A-induced apoptosis. Among the cellular regulators of apratoxin A-induced apoptosis, those targets that are particularly suitable for screening with methods of the present invention are further listed in Table 2.

TABLE 1
Apratoxin A-Modulating Polynucleotides Identified from cDNA Screening

| | GenBank | | |
|---|---|---|---|
| | accession | Symbol | Annotation |
| 1 | BC010200 | Fgfr1 | fibroblast growth factor receptor 1 |
| 2 | BC016623 | Etv4 | Ets variant gene 4 (E1A enhancer-binding protein, E1AF) |
| 3 | BC003818 | Rela | avian reticuloendotheliosis viral (v-rel) oncogene homolog A |
| 4 | BC003238 | Prkaca | protein kinase, cAMP dependent, catalytic, alpha |
| 5 | BC013572 | KRAS2 | Similar to v-Ki-ras2 Kirsten rat sarcoma 2 viral oncogene homolog |
| 6 | BC000160 | SFRS10 | splicing factor, arginine/serine-rich (transformer 2 Drosophila homolog) 10 |
| 7 | BC016281 | BCL2A1 | BCL2-related protein A1 |
| 8 | BC017040 | ETS2 | v-ets avian erythroblastosis virus E26 oncogene homolog 2 |
| 9 | BC005427 | Mc11 | myeloid cell leukemia sequence 1 |
| 10 | BC014830 | Map2k2 | mitogen activated protein kinase kinase 2 |
| 11 | BC018119 | RAF1 | v-raf-1 murine leukemia viral oncogene homolog 1 |
| 12 | BC009093 | Egr2 | early growth response 2 |
| 13 | BC027258 | BCL2 | B-cell CLL/lymphoma 2 |
| 14 | BC004642 | Kras2 | Similar to v-Ki-ras2 Kirsten rat sarcoma 2 viral oncogene homolog |
| 15 | BC005645 | Etv1 | Ets variant gene 1 |
| 16 | BC019503 | Bcl1 1b | Similar to B-cell lymphoma/leukaemia 11B |
| 17 | BC005468 | H2afx | H2A histone family, member X |
| 18 | BC026151 | Akt2 | thymoma viral proto-oncogene 2 |
| 19 | BC000953 | RIOK2 | RIO kinase 2 |
| 20 | BC003871 | Rras2 | related RAS viral r-ras oncogene homolog 2 |
| 21 | BC009014 | GDAP1L1 | hypothetical protein MGC3129 similar to ganglioside-induced |
| | | | differentiation-associated protein |
| 22 | BC003710 | Rbmx | RNA binding motif protein, X chromosome |
| 23 | BC021646 | Ppp1cc | Protein phosphatase 1, catalytic subunit, gamma isoform 1 |
| 24 | BC010588 | Ets | Ms E26 avian leukemia oncogene (Ets) |
| 25 | BC026953 | 0710007A14Rik | RIKEN cDNA 0710007A14 gene |
| 26 | BC019881 | CLK3 | CDC-like kinase 3 |
| 27 | BC027372 | 3100004P22Rik | RIKEN cDNA 3100004P22 gene |
| 28 | BC032191 | Cherp | calcium homeostasis endoplasmic reticulum protein |
| 29 | BC034200 | Pbxip1 | Ms pre-B cell leukemia transcription factor interacting protein |
| 30 | BC010683 | 3110005P07Rik | associated with Prkcl1 |
| 31 | BC017729 | EBAG9 | estrogen receptor binding site associated, antigen, 9 |
| 32 | BC023781 | Map3k3 | mitogen-activated protein kinase kinase kinase 3 |
| 33 | BC019268 | HRMT1L2 | HMT1 hnRNP methyltransferase-like 2 (S. cerevisiae) |
| 34 | NM_005239 | ETS2 | Homo sapiens v-ets erythroblastosis virus E26 oncogene homolog 2 (avian) |
| | | | (ETS2), mRNA |
| 35 | NM_004316 | ASCL1 | Homo sapiens achaete-scute complex-like 1 (Drosophila) (ASCL1), mRNA |
| 36 | NM_004902 | RNPC2 | Homo sapiens RNA-binding region (RNP1, RRM) containing 2 (RNPC2), |
| | | | mRNA |
| 37 | NM_003670 | BHLHB2 | Homo sapiens basic helix-loop-helix domain containing, class B, 2 |
| | | | (BHLHB2), mRNA |
| 38 | NM_006912 | RIT1 | Homo sapiens Ric-like, expressed in many tissues (Drosophila) (RIT), |
| | | | mRNA |
| 39 | NM_032299 | MGC2714 | Homo sapiens hypothetical protein MGC2714 (MGC2714), mRNA |
| 40 | NM_003977 | AIP | Homo sapiens aryl hydrocarbon receptor interacting protein (AIP), mRNA |
| 41 | NM_002908 | REL | Homo sapiens v-rel avian reticuloendotheliosis viral oncogene homolog |
| | | | (REL), mRNA |
| 42 | NM_001422 | ELF5 | Homo sapiens E74-like factor 5 (ets domain transcription factor) (ELF5), |
| | | | mRNA |
| 43 | NM_022963 | FGFR4 | Homo sapiens fibroblast growth factor receptor 4 (FGFR4), transcript |
| | | | variant 2, mRNA |
| 44 | NM_001167 | BIRC4 | Homo sapiens baculoviral IAP repeat-containing 4 (BIRC4), mRNA |
| 45 | NM_021975 | RELA | Homo sapiens v-rel reticuloendotheliosis viral oncogene homolog A, nuclear |
| | | | factor of kappa light polypeptide gene enhancer in B-cells 3, p65 (avian) |
| | | | (RELA), mRNA |
| 46 | NM_002401 | MAP3K3 | Homo sapiens mitogen-activated protein kinase kinase kinase 3 (MAP3K3), |
| | | | mRNA |
| 47 | NM_032241 | RPL10 | Homo sapiens ribosomal protein L10 (RPL10), mRNA |
| 48 | NM_023105 | FGFR1 | Homo sapiens fibroblast growth factor receptor 1 (fms-related tyrosine |
| | | | kinase 2, Pfeiffer syndrome) (FGFR1), transcript variant 3, mRNA |
| 49 | NM_005461 | MAFB | Homo sapiens v-maf musculoaponeurotic fibrosarcoma oncogene homolog |
| | | | B (avian) (MAFB), mRNA |
| 50 | NM_012253 | TKTL1 | Homo sapiens transketolase-like 1 (TKTL1), mRNA |
| 51 | NM_022969 | FGFR2 | Homo sapiens fibroblast growth factor receptor 2 (bacteria-expressed kinase, |
| | | | keratinocyte growth factor receptor, craniofacial dysostosis 1, Crouzon |
| | | | syndrome, Pfeiffer syndrome, Jackson-Weiss syndrome) (FGFR2), |
| | | | transcript variant 2, mRNA |
| 52 | BC006499 | HRAS | Homo sapiens Similar to v-Ha-ras Harvey rat sarcoma viral oncogene |
| | | | homolog clone MGC: 2359 IMAGE: 2819996 mRNA complete cds |
| 53 | NM_023110 | FGFR1 | Homo sapiens fibroblast growth factor receptor 1 (fms-related tyrosine |
| | | | kinase 2, Pfeiffer syndrome) (FGFR1), transcript variant 8, mRNA |

TABLE 2
Apratoxin A-Modulating Polynucleotides

| | GenBank | | |
|---|---|---|---|
| | accession | Symbol | Annotation |
| 1 | BC003238 | Prkaca | protein kinase, cAMP dependent, catalytic, alpha |
| 2 | BC000160 | SFRS10 | splicing factor, arginine/serine-rich (transformer 2 |
| | | | Drosophila homolog) 10 |
| 3 | BC009093 | Egr2 | early growth response 2 |
| 4 | BC000953 | RIOK2 | RIO kinase 2 |
| 5 | BC009014 | GDAP1L1 | hypothetical protein MGC3129 similar to ganglioside- |
| | | | induced differentiation-associated protein |
| 6 | BC003710 | Rbmx | RNA binding motif protein, X chromosome |
| 7 | BC021646 | Ppp1cc | Protein phosphatase 1, catalytic subunit, gamma isoform 1 |
| 8 | BC026953 | 0710007A14Rik | RIKEN cDNA 0710007A14 gene |
| 9 | BC019881 | CLK3 | CDC-like kinase 3 |
| 10 | BC027372 | 3100004P22Rik | RIKEN cDNA 3100004P22 gene |
| 11 | BC032191 | Cherp | calcium homeostasis endoplasmic reticulum protein |
| 12 | BC010683 | 3110005P07Rik | associated with Prkcl1 |
| 13 | BC017729 | EBAG9 | estrogen receptor binding site associated, antigen, 9 |
| 14 | BC019268 | HRMT1L2 | HMT1 hnRNP methyltransferase-like 2 (S. cerevisiae) |
| 15 | NM_004316 | ASCL1 | Homo sapiens achaete-scute complex-like 1 (Drosophila) |
| | | | (ASCL1), mRNA |
| 16 | NM_004902 | RNPC2 | Homo sapiens RNA-binding region (RNP1, RRM) |
| | | | containing 2 (RNPC2), mRNA |
| 17 | NM_003670 | BHLHB2 | Homo sapiens basic helix-loop-helix domain containing, |
| | | | class B, 2 (BHLHB2), mRNA |
| 18 | NM_006912 | RIT1 | Homo sapiens Ric-like, expressed in many tissues |
| | | | (Drosophila) (RIT), mRNA |
| 19 | NM_032299 | MGC2714 | Homo sapiens hypothetical protein MGC2714 |
| | | | (MGC2714), mRNA |
| 20 | NM_003977 | AIP | Homo sapiens aryl hydrocarbon receptor interacting |
| | | | protein (AIP), mRNA |
| 21 | NM_001422 | ELF5 | Homo sapiens E74-like factor 5 (ets domain transcription |
| | | | factor) (ELF5), mRNA |
| 22 | NM_032241 | RPL10 | Homo sapiens ribosomal protein L10 (RPL10), mRNA |
| 23 | NM_012253 | TKTL1 | Homo sapiens transketolase-like 1 (TKTL1), mRNA |

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method for identifying an antitumor agent, the method comprising:
(a) screening test compounds to identify one or more modulating agents that modulate a cellular regulator of apratoxin A-induced apoptosis that is selected from the members listed in Table 2; and
(b) testing one or more of the modulating agents for antitumor cytotoxic activity.

2. The method of claim 1, wherein the cellular regulator of apratoxin A-induced apoptosis is Prkaca, RIOK2, or CLK3.

3. The method of claim 2, wherein the test compounds are screened for ability to modulate the kinase activity of the cellular regulator of apratoxin A-induced apoptosis.

4. The method of claim 1, wherein the cellular regulator of apratoxin A-induced apoptosis is Ppp 1cc.

5. The method of claim 4, wherein the test compounds are screened for ability to modulate the phosphatase activity of the cellular regulator of apratoxin A-induced apoptosis.

6. The method of claim 1, wherein the test compounds are screened for a specific binding with the cellular regulator of apratoxin A-induced apoptosis.

7. The method of claim 1, wherein the identified modulating agents down-regulate the cellular regulator of apratoxin A-induced apoptosis.

8. The method of claim 1, wherein the identified modulating agents up-regulate the cellular regulator of apratoxin A-induced apoptosis.

9. The method of claim 1, wherein the identified modulating agents modulate cellular level of the cellular regulator of apratoxin A-induced apoptosis.

10. The method of claim 1, wherein (b) comprises testing the modulating agents for ability to inhibit proliferation of a tumor cell in vitro.

11. The method of claim 10, further comprising testing the modulating agents for cytotoxicity on a non-tumor control cell.

12. The method of claim 10, wherein the tumor cell is a cultured tumor cell line.

13. The method of claim 12, wherein the tumor cell line is a human solid tumor cell line KB or LoVo.

14. The method of claim 1, wherein (b) comprises testing the modulating agents for ability to inhibit growth of a tumor in an animal.

15. The method of claim 14, wherein the animal is a mouse.

16. A method for identifying an agent that inhibits tumor cell proliferation, the method comprising:
(a) screening test compounds to identify one or more modulating agents that modulate a cellular regulator of apratoxin A-induced apoptosis that is selected from the members listed in Table 2; and
(c) detecting a reduced proliferation of a tumor cell in the presence of the modulating agents relative to proliferation of the tumor cell in the absence of the modulating agents; thereby identifying an agent that inhibit tumor cell proliferation.

17. The method of claim 16, wherein the identified modulating agents modulate an enzymatic activity of the cellular regulator of apratoxin A-induced apoptosis.

18. The method of claim 17, wherein the cellular regulator of apratoxin A-induced apoptosis is a kinase selected from the group consisting of Prkaca, RIOK2, and CLK3.

19. The method of claim 17, wherein the cellular regulator of apratoxin A-induced apoptosis is the Ppplcc phosphatase.

20. The method of claim 16, wherein the identified modulating agents modulate cellular level of the cellular regulator of apratoxin A-induced apoptosis.
